(19) United States
(12) Patent Application Publication
(10) Pub. No.: US 2019/0247582 A1
(43) Pub. Date: Aug. 15, 2019

(54) INJECTOR EMBODIMENT WITH MODULAR PISTON FOR CONSERVING STEM CELLS

(71) Applicant: Kemal Tunc TIRYAKI, Istanbul (TR)

(72) Inventor: Kemal Tunc TIRYAKI, Istanbul (TR)

(21) Appl. No.: 16/316,843

(22) PCT Filed: Apr. 12, 2017
(86) PCT No.: PCT/TR2017/050140

§ 371 Date: Jan. 10, 2019

(30) Foreign Application Priority Data
Jul. 29, 2016 (TR) 2016/10630

(51) Int. Cl. A61M 5/315; A61M 39/20; B04B 5/04

(52) U.S. Cl. CPC A61M 5/31515; A61M 39/20; A61M 2005/3101; A61M 2202/08; B04B 5/0407

(57) ABSTRACT

An injector allowing retraction/pulling of stem cells from adipose tissue via a suction tip and conservation of thereof in a tube without human touch or exposure to air, and it is characterized in that it has a movable modular piston allowing stem cells to be pulled into the tube via the suction tip, and a modular piston pin allowing the modular piston to stay in the tube and having a modular structure detachable from the modular piston.

### THE RELATED ART

The invention relates to an injector embodiment allowing stem cells to be obtained from adipose tissue without touch by human hands and confining thereof in a tube.

### THE PRIOR ART

Stem cells are found in all body tissues and organs, and in blood circulation. However, they are more abundant especially in three places. These are the umbilical cord in babies, bone marrow, and the blood circulating in veins.

It is understood that the stem cells circulating in the body can be used in the upcoming years. For this purpose, firstly, hormone is given to the patient so as to rapidly proliferate the stem cells found in the bone marrow and pass them into the blood. Afterwards, they are collected from blood with the help of a filter (apheresis) and the remaining blood is given back to the body. This method is still being used. However, the number of stem cells obtained with this method is less compared to the method of obtaining from bone marrow.

Providing the living tissue in a lower amount than the fat injection prevents wrinkles and excessive swelling on face. Moreover, since the cells remain alive in the body, usually a single injection is sufficient. In this technique, it is possible to fill the saggy face parts that became empty underneath (cheeks, upper parts of cheekbones, and around lips), with the fat taken from abdomen or hips.

However, there are some problems in the presently used methods of obtaining stem cells. For instance, the presently used method of obtaining stem cells takes very long time.

Also, since the presently used method of obtaining stem cells is expensive, people cannot use this method commonly.

One of the reasons why the presently used method of obtaining stem cells is expensive is because the operation requires expertise. Since the operation cannot be performed by any person, this also causes the operation time to get longer.

Moreover, nowadays, stem cells are obtained by collagenase enzyme. However, use of this collagenase enzyme causes problems in some countries.

In the literature research, an application is encountered with application No. EP03759345.6, entitled: “Platelet and hematopoietic stem cell production methods” relates to a method comprising application of a TPO mimetic compound to a subject, which leads to increase of HSC production in the subject. The TPO mimetic compound can be applied to a subject alone or together with a pharmaceutically accepted carrier. The TPO mimetic compound can be applied alone or can be combined with one or more additional TPO mimetic compounds and/or other agents improving stem cell mobilization from bone marrow, as well as, for example, G-CSF, SCF, IL-3, and/or Flt-3.

Another invention encountered in the literature is the invention No. EP01911175.6, entitled: “multiple-impact stem cells produced from stroma cells obtained from adipose tissue and uses thereof”. This invention relates to multiple-impact stem cells produced from stroma cells obtained from adipose tissue and their uses. The invention includes isolated adipose tissue derived stromal cells that have been induced to express at least one phenotypic characteristic of a neuronal, astroglial, hematopoietic progenitor, or hepatic cell. The invention also includes an isolated adipocyte tissue-derived stromal cell that has been dedifferentiated such that there is an absence of adipocyte phenotypic markers.

Another invention encountered in the literature is an invention with application No. EP05743325.2, and entitled “Isolation of stem/progenitor cells from amniotic membrane of umbilical cord”. The invention relates to a method for isolation of epithelial or mesenchymal stem/progenitor cells from the amniotic membrane of umbilical cord. The method comprises separating the amniotic membrane from the other components of the umbilical cord in vitro, culturing the amniotic membrane tissue under conditions allowing cell proliferation, and isolating the stem/progenitor cells from the tissue cultures.

When the above given inventions are analyzed, an improvement for solution of the above-mentioned problems in stem cell obtaining method is not encountered.

As a result, improvement is to be made in stem cell obtaining methods, and therefore novel embodiments that would eliminate the above said drawbacks and bring solutions to the problems of the prior art systems are needed.

### BRIEF DESCRIPTION OF THE INVENTION

The purpose of the invention is to provide an embodiment wherein stem cells are obtained from adipose tissue without human touch and conserved in a tube without exposure to air.

A purpose of the invention is to prevent the stem cell from contacting air by pulling/retracting the stem cell into a tube with an injector and conserving the stem cell in the same tube without transferring said stem cell into another volume or tube.

Another purpose of the invention is to eliminate the risk of infection of cells by conserving the stem cell within the tube injector by which the first retraction is made.

Another purpose of the invention is to eliminate the loss of cells by conserving the stem cell within the tube injector by which the first retraction is made.

Another purpose of the invention is to facilitate the process by conserving the stem cell within the tube injector by which the first retraction is made.

### BRIEF DESCRIPTION OF THE FIGURES

FIG. 1. is a perspective view of the injector embodiment according to the invention.

FIG. 2. is a demounted perspective view of the injector embodiment according to the invention.

FIG. 3. is a demounted perspective view of the injector embodiment according to the invention from a different angle.

FIG. 3.1 gives the close-up detailed mounted and demounted views of the modular piston head of the invention where the O-ring and O-ring slots are shown.

FIG. 4. is a separated view of the screwed part of the piston pin.

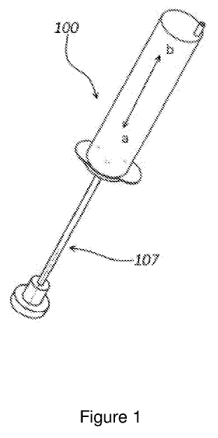

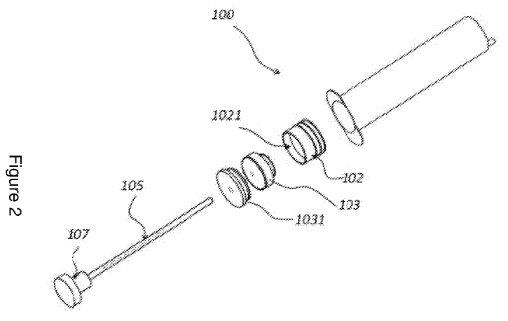

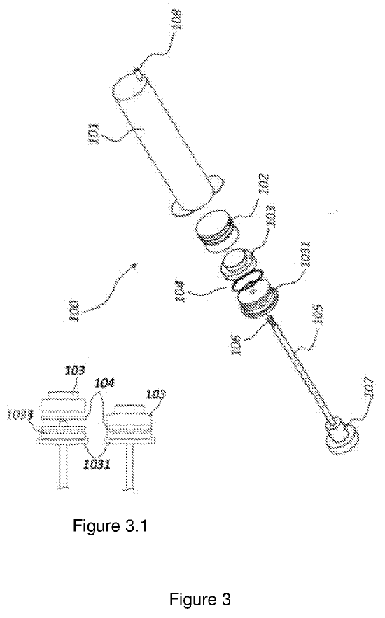

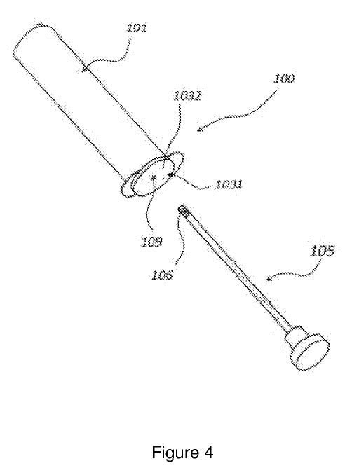

### REFERENCE NUMBERS

100. Injector embodiment
101. Tube
102. Piston nose seal
1021. Positioning space
103. Modular Piston
1031. Hollow locking piece
1032. Modular Piston Screw Socket
1033. O-ring slot
104. O-ring
105. Piston pin
106. Screwed tip
107. Piston handle
108. Suction tip
109. Piston pin slot

### DETAILED DESCRIPTION OF THE INVENTION

The invention relates to an injector embodiment (100) allowing retraction/pulling of stem cells from adipose tissue via a suction tip (108) and conservation of thereof in a tube (101) without human touch or exposure to air. The invention comprises a movable modular piston (103) allowing stem cells to be pulled into the tube (101) via said suction tip (108), and a modular piston pin (105) allowing said modular piston (103) to stay in the tube (101) and having a modular structure detachable from the modular piston (103).

It comprises screwed tips (106) allowing said modular piston pin (105) to be separated from the modular piston (103), and a modular piston screw socket (1032) formed on the upper surface of the modular piston (103) allowing detachment and fixation of said screwed tips (106). In addition, it comprises a piston handle (107) of said modular piston pin (105). On the other hand, it comprises at least one sealing O-ring (104) of said modular piston (103). It comprises a piston nose seal (102) that is confined within the tube (101) together with said modular piston (103) and has a positioning space (1021) where the modular piston (103) is to be centered. Moreover, said modular piston (103) comprises an individual hollow locking piece (1031) having a diameter that is larger than the tube (101) inner diameter.

The way of usage of the injector embodiment (100) is as follows; stem cells are sucked or pulled into the tube (101) by means of the suction tip (108) and retraction of the modular piston (103) in direction a. As a result of the retraction of the modular piston (103) in direction-a with linear motion, the piston nose seal (102) forms a high suction force with a sealed movement within the tube (101). With the stem cells being locked within the tube (101), the piston pin (105) moves freely in the piston pin slot (109) that is formed at the center of the hollow locking piece (1031) in a perpendicular manner. The modular piston pin (105) passes through the piston pin slot (109) and it is removed from the modular piston screw socket (1032) by means of the screwed tips (106). The modular piston pin (105) and the modular piston (103) that are fixed by means of screwing can thus be separated from each other via rotation. After the stem cell is pulled into the tube (101), the piston pin (105) is rotated and thus separated from the modular piston (103). In this way, the modular piston (103) piston nose seal (102) and the hollow locking piece (1031) stay fixed within the tube (101). Afterwards, the suction tip (108) is also closed by a tap and an impermeable lid so that the stem cell found within the tube (101) is prevented from contacting outside air. In order to ensure high sealing within the tube (101) and prevent the stem cell pulled into the tube (101) from leaving the tube (101) due to centrifugal force, sealing O-rings (104) are placed into the O-ring slot (1033) formed on the modular hollow locking piece (1031). Besides sealing, said sealing O-ring (104) also ensures that the hollow locking piece (1031) stays fixed against centrifugal force during centrifugation.

Said modular piston pin (105) is screwed to the modular piston (103) with a screw connection. This screwing is ensured by means of the rotating connection of the modular piston screw socket (1032) formed on the modular piston (103) and the screwed tip (106) formed on the piston pin (105). After the stem cell is pulled into the tube (101), said piston pin is removed by rotation before centrifuge operation starts. In this way, the length of the injector (100) would be reduced so that it can be placed into the centrifuge. However, this connection can also be made by a claw, interlocking, or other technique of fixing.

1. An injector allowing retraction/pulling of adipose tissue via a suction tip, conserving thereof in a tube without human touch or exposure to air, and ultimately centrifuging thereof to obtain oil-based stem cells, and it is characterized in that; it comprises:
a movable modular piston allowing adipose tissue to be pulled into the tube via said suction tip by means of a piston nose seal attached at the tip thereof,
a modular piston pin allowing back-and-forth movement of said modular piston within the tube, and having detachable characteristic so as to allow the tube to be detachable from the modular piston to enter into the centrifuge device after the adipose tissue is pulled into the tube,
an O-ring that fits into an O-ring slot formed on an independent hollow locking piece in order ensure that said independent hollow locking piece found on said modular piston can act as a lock during centrifugation, or in other words, to prevent the adipose tissue and separated stem cells found within the tube from leaving the tube during centrifugation.

2. The injector according to claim 1, and it is characterized in that it comprises a screwed tip allowing said modular piston pin to be separated from said modular piston.

3. The injector according to claim 1, and it is characterized in that it comprises a modular piston screw socket allowing connection of the screwed tip found at the tip of said modular piston pin with the modular piston and detachment of the same before centrifugation.

4. The injector according to claim 1, and it is characterized in that a piston pin slot is formed at the middle axis and vertical to the independent hollow locking piece so that said screwed tip can pass through the independent hollow locking piece so that said screwed tip can be fixed to the modular piston and detached when the operation ends.

5. The injector according to claim 1, and it is characterized in that said modular piston pin comprises a piston handle.

6. The injector according to claim 1, and it is characterized in that it comprises a replaceable piston nose seal that is confined within the tube together with said modular piston and has a positioning space where the modular piston is to be centered.

7. The injector according to claim 1, and it is characterized in that said modular piston comprises a hollow locking piece having a diameter that is larger than the tube inner diameter, and an O-ring slot in which the O-ring piece can fit in order to ensure locking for the purpose of preventing the liquid found within the tube from leaving the tube during centrifugation.
